SEQUENCE LISTING (2 sequences)

SEQ ID NO: 1 - DNA, 3400 bp, Homo sapiens
attcctcttcataatgcatgctcttttggtcatgctgaagtagtcaatctccttttgcgacatggtgcagaccccaatgctcgagataattggaattatactcctctccatgaagctgcaattaaaggaaagattgatgtttgcattgtgctgttacagcatggagctgagccaaccatccgaaatacagatggaaggacagcattggatttagcagatccatctgccaaagcagtgcttactggtgaatataagaaagatgaactcttagaaagtgccaggagtggcaatgaagaaaaaatgatggctctactcacaccattaaatgtcaactgccacgcaagtgatggcagaaagtcaactccattacatttggcagcaggatataacagagtaaagattgtacagctgttactgcaacatggacgtgatgtccatgctaaagataaaggtgatctggtaccattacacaatgcctgttcttatggtcattatgaagtaactgaacttttggtcaagcatggtggctgtgtaaatgcaatggacttgtggcaattcactcctcttcatgaggcagcttctaagaacagggttgaagtatgttctcttctcttaagttatggtgcagacccaacactgctcaattgtaagaataaaagtgctatagacttggctcccacaccacagttaaaagaaagattagcatatgaatttaaaggccactcgttgctgcaagctgcacgagaagctgatgttactcgaatcaaaaaacatctctctctggaaatggtgaatttcaagcatcctcaaacacatgaaacagcattgcattgtgctgctgcatctccatatcccaaaagaaagcaaatatgtgaactgttgctaagaaaaggagcaaacatcaatgaaaagactaaagaattcttgactcctctgcacgtggcatctgagaaagctcataatgatgttgttgaagtagtggtgaaacatgaagcaaaggttaatgctctggataatcttggtcagacttctctacacagagctgcatattgtggtcatctacaaacctgccgcctactcctgagctatgggtgtgatcctaacattatatcccttcagggctttactgctttacagatgggaaatgaaaatgtacagcaactcctccaagagggtatctcattaggtaattcagaggcagacagacaattgctggaagctgcaaaggctggagatgtcgaaactgtaaaaaaactgtgtactgttcagagtgtcaactgcagagacattgaagggcgtcagtctacaccacttcattttgcagctgggtataacagagtgtccgtggtggaatatctgctacagcatggagctgatgtgcatgctaaagataaaggaggccttgtacctttgcacaatgcatgttcttacggacattatgaagttgcagaacttcttgttaaacatggagcagtagttaatgtagctgatttatggaaatttacacctttacatgaagcagcagcaaaaggaaaatatgaaatttgcaaacttctgctccagcatggtgcagaccctacaaaaaaaaacagggatggaaatactcctttggatcttgttaaagatggagatacagatattcaagatctgcttaggggagatgcagctttgctagatgctgccaagaagggttgtttagccagagtgaagaagttgtcttctcctgataatgtaaattgccgcgatacccaaggcagacattcaacacctttacatttagcagctggttataataatttagaagttgcagagtatttgttacaacacggagctgatgtgaatgcccaagacaaaggaggacttattcctttacataatgcagcatcttacgggcatgtagatgtagcagctctactaataaagtataatgcatctctcaatgccacggacaaatgggctttcacacctttgcacgaagcagcccaaaagggacgaacacagctttgtgctttgttgctagcccatggagctgacccgactcttaaaaatcaggaaggacaaacacctttagatttagtttcagcagatgatgtcagcgctcttctgacagcagccatgcccccatctgctctgccctcttgttacaagcctcaagtgctcaatggtgtgagaagcccaggagccactgcagatgctctctcttcaggtccatctagcccatcaagcctttctgcagccagcagtcttgacaacttatctgggagtttttcagaactgtcttcagtagttagttcaagtggaacagagggtgcttccagtttggagaaaaaggaggttccaggagtagattttagcataactcaattcgtaaggaatcttggacttgagcacctaatggatatatttgagagagaacagatcactttggatgtattagttgagatggggcacaaggagctgaaggagattggaatcaatgcttatggacataggcacaaactaattaaaggagtcgagagacttatctccggacaacaaggtcttaacccatatttaactttgaacacctctggtagtggaacaattcttatagatctgtctcctgatgataaagagtttcagtctgtggaggaagagatgcaaagtacagttcgagagcacagagatggaggtcatgcaggtggaatcttcaacagatacaatattctcaagattcagaaggtttgtaacaagaaactatgggaaagatacactcaccggagaaaagaagtttctgaagaaaaccacaaccatgccaatgaacgaatgctatttcatgggtctccttttgtgaatgcaattatccacaaaggctttgatgaaaggcatgcgtacataggtggtatgtttggagctggcatttattttgctgaaaactcttccaaaagcaatcaatatgtatatggaattggaggaggtactgggtgtccagttcacaaagacagatcttgttacatttgccacaggcagctgctcttttgccgggtaaccttgggaaagtctttcctgcagttcagtgcaatgaaaatggcacattctcctccaggtcatcactcagtcactggtaggcccagtgtaaatggcctagcattagctgaatatgttatttacagaggagaacaggcttatcctgagtatttaattacttaccagattatgaggcctgaaggtatggtcgatggataaatagttattttaagaaactaattccactgaacctaaaatcatcaaagcagcagtggcctctacgttttactcctttgctgaaaaaaaatcatcttgcccacaggcctgtggcaaaaggataaaaatgtgaacgaagtttaacattctgacttgataaagctttaataatgtacag

SEQ ID NO: 2 - protein, 1074 aa, Homo sapiens
IPLHNACSFGHAEVVNLLLRHGADPNARDNWNYTPLHEAAIKGKIDVCIVLLQHGAEPTIRNTDGRTALDLADPSAKAVLTGEYKKDELLESARSGNEEKMMALLTPLNVNCHASDGRKSTPLHLAAGYNRVKIVQLLLQHGRDVHAKDKGDLVPLHNACSYGHYEVTELLVKHGGCVNAMDLWQFTPLHEAASKNRVEVCSLLLSYGADPTLLNCKNKSAIDLAPTPQLKERLAYEFKGHSLLQAAREADVTRIKKHLSLEMVNFKHPQTHETALHCAAASPYPKRKQICELLLRKGANINEKTKEFLTPLHVASEKAHNDVVEVVVKHEAKVNALDNLGQTSLHRAAYCGHLQTCRLLLSYGCDPNIISLQGFTALQMGNENVQQLLQEGISLGNSEADRQLLEAAKAGDVETVKKLCTVQSVNCRDIEGRQSTPLHFAAGYNRVSVVEYLLQHGADVHAKDKGGLVPLHNACSYGHYEVAELLVKHGAVVNVADLWKFTPLHEAAAKGKYEICKLLLQHGADPTKKNRDGNTPLDLVKDGDTDIQDLLRGDAALLDAAKKGCLARVKKLSSPDNVNCRDTQGRHSTPLHLAAGYNNLEVAEYLLQHGADVNAQDKGGLIPLHNAASYGHVDVAALLIKYNASLNATDKWAFTPLHEAAQKGRTQLCALLLAHGADPTLKNQEGQTPLDLVSADDVSALLTAAMPPSALPSCYKPQVLNGVRSPGATADALSSGPSSPSSLSAASSLDNLSGSFSELSSVVSSSGTEGASSLEKKEVPGVDFSITQFVRNLGLEHLMDIFEREQITLDVLVEMGHKELKEIGINAYGHRHKLIKGVERLISGQQGLNPYLTLNTSGSGTILIDLSPDDKEFQSVEEEMQSTVREHRDGGHAGGIFNRYNILKIQKVCNKKLWERYTHRRKEVSEENHNHANERMLFHGSPFVNAIIHKGFDERHAYIGGMFGAGIYFAENSSKSNQYVYGIGGGTGCPVHKDRSCYICHRQLLFCRVTLGKSFLQFSAMKMAHSPPGHHSVTGRPSVNGLALAEYVIYRGEEAYPEYLITYQIMRPEGMVDG

(19) United States
(12) Patent Application Publication
(10) Pub. No.: US 2002/0037582 A1
(43) Pub. Date: Mar. 28, 2002

(54) POTENTIAL EFFECTOR FOR THE GRB7 FAMILY OF SIGNALLING PROTEINS

(76) Inventors: ROGER JOHN DALY, ALEXANDRIA (AU); ROBERT L. SUTHERLAND, NSW (AU)

Correspondence Address: ROTHWELL FIGG ERNST & KURZ, COLUMBIA SQUARE, SUITE 701 EAST TOWER, WASHINGTON, DC, 20004, US

(21) Appl. No.: 09/509,196

(22) PCT Filed: Sep. 23, 1998
(86) PCT No.: PCT/AU98/00795

(30) Foreign Application Priority Data
Sep. 23, 1997 (AU) PO 9388

(51) Int. Cl. C07H 21/04; C12P 21/06; C12N 15/74; C12N 15/00; C12N 15/09; C12N 15/63; C12N 15/70; C07K 5/00; C07K 7/00; C07K 16/00; C07K 17/00; A61K 38/00; C12N 5/00; C12N 5/02; C12P 21/08

(52) U.S. Cl. 435/325; 536/23.5; 435/320.1; 435/69.1; 530/324; 530/387.9; 536/24.32

(57) ABSTRACT

A novel polynucleotide molecule is disclosed which encodes a candidate effector protein for the Grb7 family of signalling proteins. Detection of the protein in a sample such as a homogenised tissue sample should provide a useful tumour marker and/or prognostic indicator for certain human cancers such as breast and prostate cancer.

### FIELD OF THE INVENTION

The present invention relates to a novel polynucleotide molecule encoding a candidate effector protein for the Grb7 family of signalling proteins. Detection of the encoded protein in a tissue sample should provide a useful tumour marker and/or prognostic indicator. Furthermore, antagonism of the interaction between Grb7 family members and the encoded protein should provide a novel treatment strategy for human diseases exhibiting aberrant receptor tyrosine kinase (RTK) signalling (e.g. cancer).

### BACKGROUND OF THE INVENTION

RTKs play a major role in the regulation of cellular growth, differentiation, motility and metabolism by converting an extracellular signal in the form of the binding of a specific hormone or growth factor to the activation of specific signalling pathways and hence modes of intracellular communication (Schilessinger and Ullrich, Neuron 9, 383-391, 1992). Activation of RTKs results in both autophosphorylation of the receptor and the phosphorylation of downstream targets on tyrosinie residues. It has become evident over the last decade that key elements in receptor-substrate and other protein-protein interactions in RTK signalling are src homology (SH)2 domains. SH2 domains are conserved modules of approximately 100 amino acids found in a wide variety of signalling molecules which bind to short tyrosine-phosphorylated peptide sequences. The specificity of interaction is determined both by the nature of the amino acids flanking the phosphotyrosine residue in the target peptide and residues in the SH2 domain which interact with these sites (Pawson, Nature 373, 573-580, 1995).

SH2-domain containing proteins can be divided into two classes: those which possess a catalytic function (e.g. the cytoplasmic tyrosine kinase c-src and the tyosine phosphatase SH-PTP2) and those which consist entirely of non-catalytic protein domains (eg Grb2), the adaptor sub-class. The function of the latter class is to link separate catalytic subunits to a tyrosine-phosphorylated receptor or signalling intermediate, and other non-catalytic protein modules are often involved in these interactions. For example, SH3 and WW domains (conserved regions of approximately 50 and 40 amino acids, respectively) bind proline-rich peptide ligands, and pleckstrin homology domains (approximately 100 amino acids) interact with both specific phospholipid and protein targets (Pawson, 1995 supra).

The Grb7 family represents a family of SH2 domain-containing adaptors which currently contains three members: CGrb7. 10 and 14 (Margolis et al., Proc. Natl. Acad. Sci. USA 89, 8894-8898, 1992: Stein et al. EMBO J 13, 1331-1340, 1994: Ooi et al. Oncogene 10, 1621-1630, 1995: Daly et al. J. Biol. Chem. 271, 12502-12510, 1996). These proteins share a common overall architecture, consisting of an N-terminal region containing a highly conserved proline-rich decapeptide motif, a central region harbouring a PH domain and a C-terminial SH2 domain. The central region of approximately 300 amino acids bears significant homology to the C. elegans protein mig10, which is required for long range neuronal migration in embryos, otherwise the Grb7 family and mig10 are structurally distinct. However, they exhibit differences in both SH2 selectivity towards RTKs (Janes et al, J. Biol. Chem. 272, 8490-8497, 1997) and tissue distribution. The family has therefore evolved to link particular receptors to downstream effectors in a tissue-specific manner. Interestingly, the genes encoding this family appear to have co-segregated with ERBB family genes during evolution. Thus GRB7, 10 and 14 are linked to ERBB2, ERBB1 (epidermal growth factor receptor) and ERBB4, respectively (Stein et al 1994 supra; Ooi et al, 1995 supra: Baker et al. Genomics 36, 218-220, 1996). The juxtaposition of GRB7 and ERBB2 leads to common co-amplification in human breast cancers, and since the two gene products are functionally linked, likely up-regulation of an undefined erbB2 signalling pathway. Furthermore, GRB14 also exhibits differential expression in human breast cancers (Daly et al, 1996 supra). These two proteins may therefore modulate RTK signalling in this disease.

In order to identify proteins which bind to this family and therefore identify candidate effectors, we performed a genetic screen using the yeast two hybrid system and Grb14 “bait”. This application describes the cloning and characterization of a novel interacting protein, currently designated 2.2412.

### DISCLOSURE OF THE INVENTION

Thus, in a first aspect, the present invention provides an isolated polynucleotide molecule encoding a candidate effector protein for the Grb7 family of signalling proteins, wherein the polynucleotide molecule comprises a nucleotide sequence having at least 75%, sequence identity to that shown as SEQ ID NO: 1.

Preferably, the polynucleotide molecule comprises a nucleotide sequence having at least 85%, more preferably at least 95%, sequence identity to that shown as SEQ ID NO: 1. Most preferably, the polynucleotide molecule comprises a nucleotide sequence encoding a polypeptide comprising an amino acid sequence substantially corresponding to that shown as SEQ ID NO: 2.

In a preferred embodiment of the invention of the first aspect, the polynucleotide molecule comprises a nucleotide sequence which substantially corresponds to that shown as SEQ ID NO: 1.

The polynucleotide molecule may be a dominant negative mutant which encodes a gene product causing an altered phenotype by, for example, reducing or eliminating the activity of endogenous effector proteins of the Grb7 family of signalling proteins.

The polynucleotide molecule may be incorporated into plasmids or expression vectors (including viral vectors), which may then be introduced into suitable host cells such as bacterial, yeast, insect and mammalian host cells. Such host cells may be used to express the protein encoded by the polynucleotide molecule.

Accordingly, in a second aspect, the present invention provides a host cell transformed with the polynucleotide molecule of the first aspect.

In a third aspect, the present invention provides a method of producing a protein, comprising culturing the host cell of the second aspect under conditions suitable for the expression of the polynucleotide molecule and optionally recovering the protein.

Preferably, the host cell is mammalian or of insect origin. Where the cell is mammalian, it is presently preferred that it be a Chinese hamster ovary (CHO) cell or human embryonic kidney (HEK) 293 cell. Where the host cell is of insect origin, it is presently preferred that it be an insect Sf9 cell.

In a fourth aspect, the present invention provides a purified protein encoded by the polynucleotide molecule of the first aspect.

In a preferred embodiment of this aspect, the purified protein comprises an amino acid sequence substantially corresponding to that shown as SEQ ID NO: 2.

In a fifth aspect, the present invention provides a fusion protein comprising an amino acid sequence substantially corresponding to that shown as SEQ ID NO: 2.

Fusion proteins according to the fifth aspect may include an N-terminal fragment of a protein such as β-galactosidase to assist in the expression and selection of host cells expressing candidate effector protein, or may include a functional fragment of any other suitable protein to confer additional activity(ies).

In a sixth aspect, the present invention provides all antibody or fragment thereof which specifically binds to the protein of the fourth aspect.

The antibody may be monoclonal or polyclonal, however, it is presently preferred that the antibody is a monoclonal antibody. Suitable antibody fragments include Fab, F(ab′)₂ and scFv.

In a seventh aspect, the present invention provides an oligonucleotide probe comprising a nucleotide sequence of at least 12 nucleotides, the oligonucleotide probe comprising a nucleotide sequence such that the olignucleotide probe selectively hybridises to the polynucleotide molecule of the first aspect under high stringency conditions (Sambrook et al., Molecular Cloning: a Laboratory Manual. Second Edition. Cold Spring Harbor Laboratory Press).

In a preferred embodiment of this aspect, the oligonucleotide probe is labelled. In a further preferred embodiment of this aspect, the oligonucleotide probe comprises a nucleotide sequence of at least 18 nucleotides.

In an eighth aspect, the present invention provides a method of detecting in a sample the presence of all effector protein for the Grb7 family of proteins, the method comprising reacting the sample with an antibody or fragment thereof the sixth aspect, and detecting the binding of the antibody or fragment thereof.

The method of the eighth aspect may be conducted using any immunoassays well known in the art (e.g. ELISA). The sample may be, for example, a cell lysate or homogenate prepared from a tissue biopsy.

In a ninth aspect, the present invention provides a method of detecting in a sample the presence of mRNA encoding an effector protein for the Grb7 family of proteins, the method comprising reacting the sample with an oligonucleotide probe of the seventh aspect, and detecting the binding of the probe.

The method of the ninth aspect may be conducted using any hybridisation assays well known in the art (e.g. Northern blot). The sample may be a poly(A) RNA preparation or homogenate prepared from a tissue biopsy.

Grb7 family proteins exhibit differential expression in certain human cancers (particularly breast and prostate cancer) and may therefore be involved in tumour progression. Detection of the protein encoded by the cDNA 2.2412 in a sample should provide a useful tumour marker and/or prognostic indicator for these cancers. Furthermore, the interaction of Grb7 family members with 2.2412 may provide a novel target for therapeutic intervention.

It is to be understood that methods of detecting suitable agonists and methods of therapy utilising detected agonists also form part of the present invention. The term “substantially corresponds” as used herein in relation to the nucleotide sequence shown as SEQ ID NO: 1 is intended to encompass minor variations in the nucleotide sequence which due to degeneracy in the DNA code do not result in a change in the encoded protein. Further, this term is intended to encompass other minor variations in the sequence which may be required to enhance expression in a particular system but in which the variations do not result in a decrease in biological activity of the encoded protein.

The term “substantially corresponding” as used herein in relation to the amino acid sequences shown as SEQ ID NO: 2 is intended to encompass minor variations in the amino acid sequences which do not result in a decrease in biological activity of the protein. These variations may include conservative amino acid substitutions. The substitutions envisaged are:-

G, A, V, I, L, M: D, E; N, Q; S, T; K, R, H: F, Y, W, H: and

P, Nα-alkalamino acids.

The terms “comprise”, “comprises” and “comprising” as used throughout the specification are intended to refer to the inclusion of a stated step, component or feature of group of steps, components of features with or without the inclusion of a further step, component or feature or group of steps, components or features.

The invention will hereinafter be described with reference to the accompanying figure and the following, non-limiting example.

### BRIEF DESCRIPTION OF THE ACCOMPANYING FIGURE:

FIG. 1 provides the nucleotide and amino acid (single letter code) sequence of 2.2412. Numbers refer to distances in base pairs. Ankyrin-type repeat sequences are underlined. An additional repeat sequence is indicated by italics. The stop codon is represented by all asterisk. The original cDNA clone 2.2412 isolated by the two hybrid screen spans nucleotides 694-2664 of this sequence.

FIG. 2 provides a map of the 2.2412-binding region on Grb14. A. Structure of the deletion constructs used in the analysis. Ga14 DNA-BD fusion constructs encoding full length Grb14 (FL), the N-terminal (N), central region (C) and N-terminal+central region (N+C) were generated in the vector pAS2.1. B. Results of β-galactosidase activity assays following transformation of the above plasmids into yeast strain Y190 together with the original 2.2412 cDNA clone in pACT-2.

### EXAMPLE

### CLONING AND CHARACTERISATION OF 2.2412

Yeast two hybrid screen

The yeast two hybrid system exploits protein-protein interactions to reconstitute a functional transcriptional activator which can then be detected using a gene reporter system (Fields and Sternglanz. TIG. 10, 286-292, 1994). The technique takes advantage of the properties of the Ga14 protein of the yeast S. cerevisiae. The Gal4 DNA binding domain (DNA-BD) or activation domain (AD) alone are incapable of inducing transcription. However, an interaction between two proteins synthesized as DNA-BD- and AD-fusions, respectively, brings the Gal4 domains into close proximity and results in transcriptional activation of two reporter genes (HIS3 and LacZ) which can be monitored by growth on selective medium and biochemical assays.

A plasmid construct encoding a Gal4 DNA-BD-Grb14 fusion was generated as follows. The plasimid GRB14/pRcCMV_{F} containing full length GRB14 cDNA (Daly et al. 1996) was restricted with HindIII and Klenow treated to create blunt ends, and then digested with BcII to release three fragments of approximately 1.1, 4.2 and 1.7 kb. The 1.7 kb fragment was isolated and cloned into the NdeI (Klenow treated) and BamHI sites of the yeast expression vector pAS2.1 (Clontech) to generate GRB14/pAS2.1 containing an ill-frame fusion of full length Grb14 with the GAL4 DNA-BD. This construct was introduced by electroporation into the yeast strain CG1945 (MATα, ura3-52, his3-200, ade2-101, lys2-801,trp1-901, leu2-3, 112, gal4-542, gal80-538, cyh^{r}2, LYS2::GAL1_{UAS}-GAL1_{TATA}-HIS3, URA3::GAL4₁₇ₘₑᵣₛ₍ₓ₃₎-CYC1_{TATA}-lacZ) selecting for tryptophan prototrophy. The expression of the fusion protein was verified by Western blot analysis with antibodies directed against the Flag epitope and the Gal4 DNA-BD. The recipient strain was then grown to mid-log phase and a human liver cDNA library in the vector pACT2 (Clontech) introduced using the LiAc procedure (Schiestl and Gietz, Curr. Genet. 16, 339-346, 1989). Transformants were then selected for tryptophan, leucine and histidine prototrophy in the presence of 5 mM 3-aminotriazole.

From a screen of 1×10⁶ clones, 39 colonies were initially selected on synthetic complete (SC)-leu-his-trp+3AT medium and were then tested for β-galactosidase activity, 12 clones scored positive in the latter assay and were subjected to cycloheximide (CHX) curing to remove the bait plasmid by streaking out on SC-leu media containing 10 μg/ml CHX (pAS2-1 contains the CYH2 gene which restores CHX sensitivity to CG1945 cells). This enabled confirmation of the bait dependency of LacZ activation and subsequent isolation of the pACT2 plasmids encoding interacting proteins by standard methodology (Philippsen et al, Methods in Enzymology 194, 170-177). Back transformations were then performed in which these pACT2 plasmids were introduced into CG1945 strains containing the bait plasmid (GRB14/pAS2-1) or constructs encoding non-related Ga14 DNA-BD fusions in order to confirm the specificity of the interactions.

The DNA sequences of the cDNA inserts were then obtained by cycle sequencing (f-mol kit, Promega) using pACT2-specific and/or clone-specific primers. Based on their nucleotide sequences the 12 interacting clones were classified into 6 independent groups (see Table I).

TABLE I
Characterization of cDNA clones isolated by the yeast two
hybrid screen.

| | No. of | | Mean RLU | Colour intensity |
|---|---|---|---|---|
| Class | clones | Identity | (Liquid assay) | (Filter assay) |
| 1 | 6 | Nedd4 | 2.86 × 10⁶ | ++++ |
| 2 | 2 | Htk | 1.86 × 10⁵ | ++ |
| 3 | 1 | 2.2412 | 5.18 × 10⁶ | ++++ |
| 4 | 1 | Proteosome | 3.88 × 10² | +/− |
| 5 | 1 | Somatostatin | 1.45 × 10³ | +/− |
| | | receptor | | |
| 6 | 1 | L-arginine:glycine | 8.61 × 10² | +/− |
| | | amidinotransferase | | |
| The 12 clones exhibiting activation of both the HIS3 and lacZ reporter genes were divided into 6 groups by sequence analysis of their cDNA inserts. | | | | |
| # Results of β-galactosidase activity assays performed using two methodologies are shown. The liquid culture-derived method (Galacto-Light TROPIX) is more quantitative: results are given in mean relative light units (RLU) and are normalized for the protein content of the samples. Blue/white screening of the cDNA clones was also performed using a colony | | | | |
| # lift filter assay (Cloatech). The intensity of blue colour development over approximately 2h is scored from ± (very weak) to ++++ (strong). | | | | |

Six clones were partial cDNAs corresponding to Nedd4, a multidomain protein containing a calcium-dependent phospholipid binding (CaLB) domain, four WW domains and a C-terminal region homologous to the E6-AP carboxyl-terminus (Kumar et al, Biochem. Biophys. Res. Commun. 185, 1155-1161, 1992: Sudol et al J. Biol. Chem. 270, 14733-14741. 1995; Huibregtse et al Proc. Natl. Acad. Sci. USA 92, 2563-2567, 1995). The latter is likely to confer E3 ubiquitin-protein ligase activity on Nedd4. The pACT2 clones isolated encoded the CaLB domain together with the first 22 amino acids of the first WW domain.

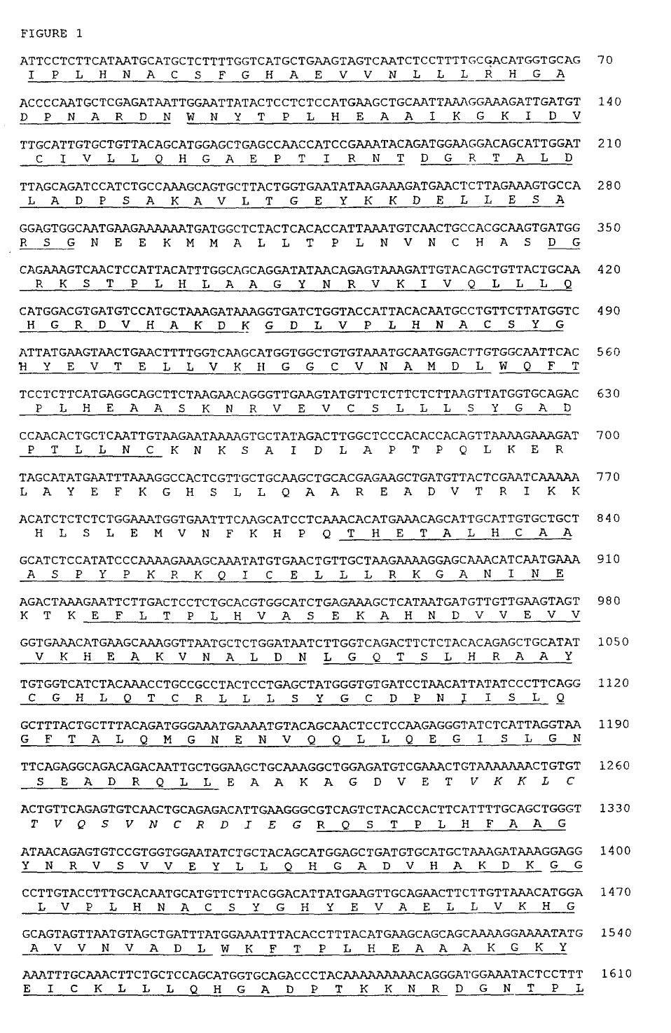

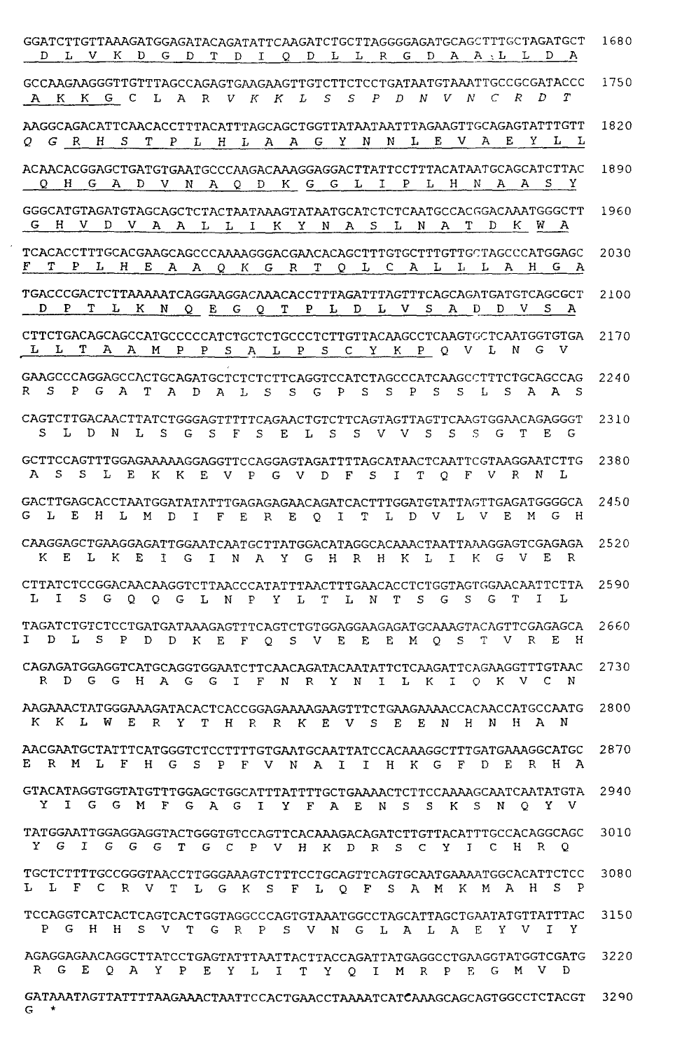

Two clones encoded the intracellular region and part of the extracellular domain of Htk, which is a RTK of the Eph family (Bennett et al J. Biol. Chem. 269, 14211-14218, 1994). The recruitment of Grb14 by Htk is of interest for two reasons. First, the expression profile of both Htk and the murine homologue myk-1 are indicative of a potential role in mammary gland development and neoplasia (Andres et al Oncogene 9, 1461-1467, 1994: Berclaz et al Biochem. Biophys. Res. Comm. 226, 869-875, 1996). Second, Eph family members may be involved in the regulation of cell migration (Tessier-Lavigne, Cell 82, 345-348, 1995), which is intriguing given the homology of the Grb7 family to the C. elegans protein mig10 (Stein et al. 1994 supra). A novel cDNA of 1971 bp, designated 2.2412. was also isolated. This clone encoded a polypeptide of 657 amino acids in frame with the Ga14 DNA-BD. The cDNA did not contain a stop codon, and this, together with the Northern analysis described below, indicated that it was incomplete. This DNA fragment was therefore used as a probe to screen a human placental cDNA library (5′ STRETCH PLUS. Clontech, in λgt10). This resulted in the isolation of two clones, designated clone 8 and clone 12. Clone 8 was approximately 2 kb and overlapped the original 2.2412 clone by 900 bp at the 3′ end. This clone provided the carboxy-terminal end of the 2.2412 protein sequence (FIG. 1). Clone 12 was approximately 3.5 kb and to date has provided an additional 692 bp of sequence information in the 5′ direction. The nucleotide and protein sequence for 2.2412 provided by these overlapping clones is shown in FIG. 1. Since a 5′ initiation codon has vet to be identified the coding sequence still appears to be incomplete.

Further characterization of 2.2412

Database searches using the 2.2412 cDNA sequence revealed significant homology with a large number of proteins containing ankyrin-like repeats. These sequences were first identified as homologous regions between certain cell cycle regulatory proteins and the Drosophila protein Notch (Breeden and Nasmyth, Nature 329, 651-654, 1987) but subsequently they have been identified in a wide variety of other proteins where they are thought to function in protein-protein interactions (Bork, Proteins 17, 363-374, 1993). Subsequent analysis of the protein sequence identified 18 consecutive ankyrin repeats and an additional repetitive element (FIG. 1). The ankyrin repeat region is followed by a stretch of approximately 40 amino acids rich in serine residues. The remaining C-terminal region has a relatively high content of charged amino acids.

Northern analysis of 2.2412 mRNA expression

Northern blot analysis of multiple tissue northerns (Clontech) was performed using the original 2.2412 cDNA as a probe. This resulted in the detection of a single mRNA transcript of approximately 7 kb in all tissues examined with the exception of the kidney. Expression was particularly high in skeletal muscle and placenta. The size of this transcript compared to that of the 2.2412 clone indicates that the latter represents only a partial cDNA.

Genomic localization of the 2.2412 gene

Fluorescence in situ hybridization of the original 2.2412 cDNA to normal metaphases (Baker et al. 1996 supra) and reference to the FRA10A fragile site at 10q23.32 localized the gene to between chromosome 10q23.2 and proximal 10q23.32. Interestingly, deletions in the 10q22-25 region of chromosome 10 have been detected in a variety of human cancers including breast, prostate, renal, small cell lung and endometrial carcinomas, glioblastoma multiforme, melanoma and meningiomas, suggesting the presence of one or more tumour suppressive loci in this region (Li et al, Science 275, 1943-1947, 1997: Steck et al, Nature Genetics 15, 356-362, 1997, and references therein). Two candidate tumour suppressor genes have been identified in this region (MMAC1/PTEN and MXI1. Li et al 1997 supra: Steck et al 1997 supra; Albarosa et al, Hum. Genet. 95, 709-711, 1995).

Analysis of the interaction between 2.2412 and Grb7 family members

cDNAs encoding the full length and N- and C-terminal regions of the original 2.2412 cDNA clone (nucleotides 694-2664, 694-1614 and 1615-2664 of the sequence shown in FIG. 1, respectively) were cloned into the vector pGEX4T2 (Pharmacia). The full length construct was generated by subcloning from the pACT2 clone as a NdeI fragment, whereas the shorter constructs were synthesized by directional cloning of PCR products. The corresponding GST-fusion proteins were purified from IPTG-induced bacterial cultures using glutathione-agarose beads (Smith and Johnson, Gene 67, 31-40, 1988). These immobilized fusion proteins were then incubated with lysates from cells expressing Flag epitope-tagged Grb14 (Daly et al. 1996 supra) or human breast cancel cells expressing high levels of Grb7 (SK-BR-3: Stein et al. 1994) as described previously (Daly et al. 1996). Following washing, bound proteins were detected by Western blot analysis. The results indicated that 2.2412 bound specifically to both Grb14 and Grb7 in vitro, and that the N-terminal fusion protein bound more strongly than that derived from the C-terminus. These data, obtained using a different methodology for detecting protein-protein interactions to the yeast two hybrid system, confirm that 2.2412 interacts with Grb14. Furthermore, 2.2412 also binds Grb7. Consequently 2.2412 appears to represent a general effector for the Grb7 family.

Mapping of the 2.2412 binding region Grb14

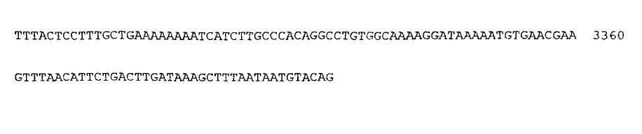

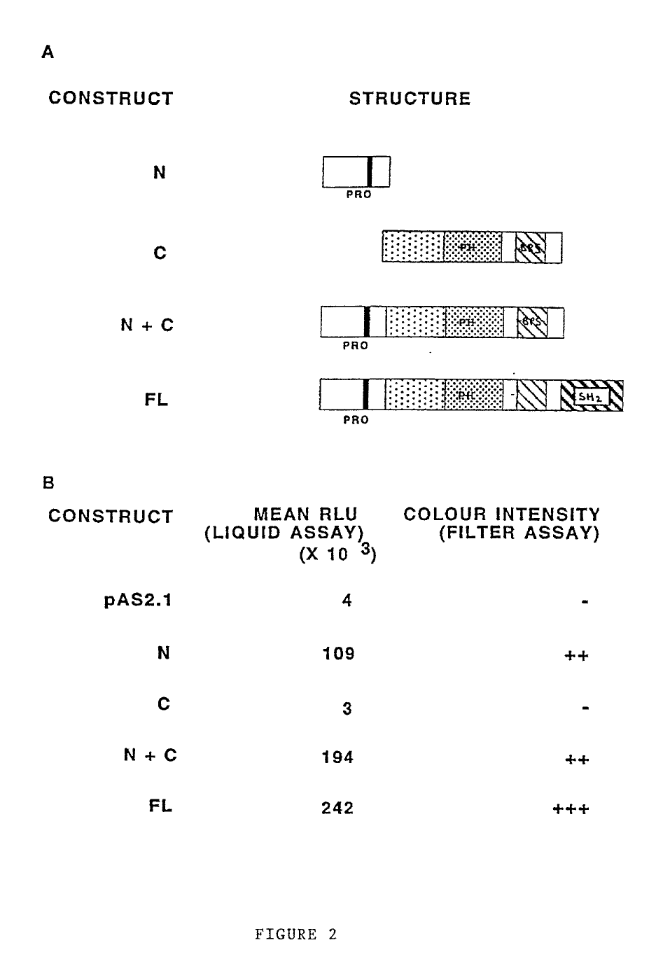

In order to identify the region of Grb14 that interacts with 2.2412. a series of Grb14 deletion mutants were generated by cloning PCR fragments synthesized using the appropriate flanking primers into the vector pAS2.1. These fragments spanned the following regions: N-terminus (“N”. amino acids 1-110), the central region (“C”) encompassing the mig10 homology and the “between PH and SH2” (BPS) domain (amino acids 110-437) and the N-terminal and central regions (“N+C”, amino acids 1-437). These plasmids were individually transformed into the yeast strain Y190 (MATα, ura3-52, his3-200, ade2-101, lys2-801, trp1-901, leu2-3, 112, gal4Δ, gal80Δ, cyh^{r}2, LYS2::GAL1_{UAS}-HIS3_{TATA}-HIS3, URA3::GAL1_{UAS}-GAL1_{TATA}-lacZ) and expression of the appropriately sized Gal4 DNA-BD fusion proteins confirmed by Western blotting. Following transformation of the resulting yeast strains with the original 2.2412 CDNA clone in pACT-2, the strength of the interaction was determined by either liquid- or filter-based 3-galactosidase assays. The results are presented in FIG. 2, and demonstrate that the N-terminal region of Grb14 is not only required, but is also sufficient, for binding 2.2412. This supports the hypothesis that 2.2412 represents a general effector for the Grb7 family, since the N-terminal region of these proteins contains a highly conserved proline-rich motif which may mediate this interaction.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An isolated polynucelotide molecule encoding a candidate effector protein for the Grb7 family of signalling proteins, wherein the polynucleotide molecule comprises a nucleotide sequence having at least 75% sequence identity to that shown as SEQ ID NO: 1.

2. A polynucleotide molecule according to claim 1, wherein the polynucleotide molecule comprises a nucleotide sequence having at least 85% sequence identity to that shown as SEQ ID NO: 1.

3. A polynucleotide molecule according to claim 1, wherein the polynucleotide molecule comprises a nucleotide sequence having at least 95% sequence identity to that shown as SEQ ID NO: 1.

4. A polynucleotide molecule according to claim 1, wherein the polynucleotide molecule comprises a nucleotide sequence which substantially corresponds to that shown as SEQ ID NO: 1.

5. A host cell transformed with a polynucleotide molecule according to any one of the preceding claims.

6. A host cell according to claim 5, wherein the host cell is a mammalian, insect, yeast or bacterial host cell.

7. A method of producing a protein, comprising culturing the host cell of claim 5 or 6 under conditions suitable for the expression of the polynucleotide molecule and optionally recovering the protein.

8. A purified protein encoded by a polynucleotide molecule according to any one of claims 1 to 4.

9. A purified protein according to claim 8, wherein the protein comprises an amino acid sequence substantially corresponding to that shown as SEQ ID NO: 2.

10. A fusion protein comprising an amino acid sequence substantially corresponding to that shown as SEQ ID NO: 2.

11. An antibody or fragment thereof which specifically binds to a protein according to claim 8 or 9.

12. An oligonucleotide probe comprising a nucleotide sequence of at least 12 nucleotides, the oligonucleotide probe comprising a nucleotide sequence such that the oligonucleotide probe selectively hybridises to the polynucleotide molecule of any one of claims 1 to 4 under high stringency conditions.

13. An oligonucleotide probe according to claim 12, wherein the oligonucleotide probe comprises a nucleotide sequence of at least 18 nucleotides.

14. A method of detecting in a sample the presence of an effector protein for the Grb7 family of proteins, the method comprising reacting the sample with an antibody or fragment thereof according to claim 11.

15. A method of detecting in a sample the presence of mRNA encoding an effector protein for the Grb7 family of proteins, the method comprising reacting the sample with an oligonucleotide probe of claim 12 or 13.
